(19) United States
(12) Patent Application Publication
(10) Pub. No.: US 2026/0283453 A1
(43) Pub. Date: Sep. 24, 2026

(54) SURGICAL INSTRUMENT WITH DISTAL VISUALIZATION AND PROXIMAL OPTICAL FIDUCIALS

(71) Applicant: Dearborn Capital Management LLC, Miami, FL (US)

(72) Inventor: Krzysztof SIEMIONOW, Miami, FL (US)

(21) Appl. No.: 19/574,757

(22) Filed: Mar. 23, 2026

Related U.S. Application Data
(60) Provisional application No. 63/776,027, filed on Mar. 22, 2025.

(51) Int. Cl. A61B 1/05; A61B 1/00; A61B 1/06; A61B 34/20; A61B 90/00

(52) U.S. Cl. CPC A61B 1/05; A61B 1/00057; A61B 1/0661; A61B 34/20; A61B 90/39; A61B 2034/2057

(57) ABSTRACT

A surgical instrument includes an elongated body extending from a proximal region to a distal working region defining a predetermined distal working point. A vision module positioned proximal to the distal working point includes at least one camera and at least one light source for acquiring live video of tissue adjacent the distal working region. A fiducial assembly mounted at or near the proximal region includes optical tracking elements arranged in a predefined geometry detectable by an external optical navigation system. One or more reference features provided by cooperation of the fiducial assembly and the elongated body, or by a fixed manufactured structural relationship between them, establish a repeatable spatial relationship between a fiducial coordinate frame and at least one of a camera coordinate frame and a distal-working-point coordinate frame.

### FIELD OF THE INVENTION

The present invention relates generally to surgical instruments for minimally invasive procedures.

More particularly, the invention relates to surgical instruments having a distal vision module and a proximally mounted fiducial assembly detectable by an external navigation system, and to associated systems and methods for calibrating, maintaining, and using a spatial relationship between the fiducial assembly and a distal camera and/or distal working point.

The invention is applicable, by way of example, to neurosurgery, spine surgery, orthopedic surgery, ear, nose, and throat surgery, interventional radiology, and pain management procedures.

### BACKGROUND OF THE INVENTION

Minimally invasive surgical procedures can reduce patient trauma, recovery time, and scarring, but they often require a surgeon to operate within a limited field with reduced direct visualization of tissue and constrained instrument access.

Surgical visualization systems and surgical navigation systems can each provide useful information during such procedures. However, visual information from a separate endoscope or other camera system may not be intrinsically correlated with the position and orientation of the specific surgical instrument interacting with tissue. As a result, a surgeon may be required to mentally reconcile multiple streams of information while operating in a confined anatomical corridor.

Instruments having local visualization capability may improve the ability to inspect tissue adjacent a working tip, while tracked instruments may improve positional awareness relative to patient anatomy. Nonetheless, a further challenge arises when a tracked structure is located away from the distal working region of the instrument. In such arrangements, reliable use of tracked pose information may depend on maintaining a known spatial relationship between the tracked structure and the distal camera and/or distal working point.

Accuracy may be affected by factors such as assembly tolerances, mounting configuration, partial occlusion of fiducials, re-seating of components, or deviations detected during use. There remains a need for a surgical instrument and associated navigation workflow that provide direct distal visualization together with tracked pose information that is consistently correlated to the distal camera and/or distal working point of the instrument.

There remains a further need for such a system to support calibration, recalibration, confidence assessment, and guided presentation of navigational information in a manner that reduces surgeon cognitive load while preserving broad applicability across different instrument forms and procedure types.

### SUMMARY OF THE INVENTION

In one aspect, the invention provides a surgical instrument comprising an elongated body extending from a proximal region to a distal working region and defining a predetermined distal working point at the distal working region. The instrument further comprises a vision module carried by the elongated body at a position proximal to the distal working point, and a fiducial assembly mounted at or near the proximal region of the elongated body. The vision module may comprise at least one camera and at least one light source configured to acquire image data of tissue adjacent the distal working point. The fiducial assembly may comprise a plurality of optical tracking elements arranged in a predefined geometry detectable by an external optical navigation system.

In some embodiments, the instrument further comprises one or more reference features provided by cooperation of the fiducial assembly and the instrument body, or by a fixed manufactured structural relationship between the fiducial assembly and the instrument body, the one or more reference features being configured to establish a repeatable spatial relationship between a fiducial coordinate frame and at least one of a camera coordinate frame and a distal-working-point coordinate frame. In embodiments in which the geometry between the fiducial assembly and the distal working region is fixed, the spatial relationship may be represented by a stored transform. In embodiments in which one or more portions between the fiducial assembly and the distal working region are movable, adjustable, or selectively flexible, the spatial relationship may be represented by one of a plurality of stored state-associated transforms or by a transform updated according to a sensed, commanded, or otherwise determined instrument state.

In certain embodiments, the optical tracking elements comprise passive retroreflective markers, active infrared emitters, or a combination thereof. The fiducial assembly may be permanently integrated with the instrument body or may be coupled thereto as a removable module. The fiducial assembly may be located, by way of example, on a proximal handle portion, a shaft collar, a dorsal bridge, a lateral frame, or a proximal extension of the instrument.

In some embodiments, the vision module is recessed from the distal-most working point of the instrument, for example by a distance selected to protect the camera from direct contact with tissue and debris while still keeping the distal working point and adjacent anatomy within the camera field of view. In some embodiments, the camera is a miniature CCD or CMOS camera, optionally on the order of 1 mm or less in one or more dimensions. One or more LEDs may be positioned adjacent the camera to illuminate the surgical field. In further embodiments, the vision module comprises multiple cameras configured for stereoscopic imaging and local depth estimation. The optical orientation of the camera may be fixed relative to the elongated body or may be adjustable; where adjustable, the current angular state may be constrained, sensed, or otherwise determined so that a corresponding camera coordinate frame remains known or derivable during use. Likewise, where variable optical settings affect camera calibration parameters, the current calibration parameters corresponding to the active setting may be stored, retrieved, or determined during use.

In another aspect, the invention provides a surgical system comprising the surgical instrument, an external optical navigation system configured to detect the fiducial assembly and to generate marker observations and/or determine a pose of the fiducial assembly in a navigation coordinate frame, and a processor configured to receive the pose of the fiducial assembly or the marker observations, apply one or more stored transforms between a fiducial coordinate frame associated with the fiducial assembly and at least one of the camera coordinate frame and the distal-working-point coordinate frame, and derive a pose of at least one of the camera coordinate frame and the distal-working-point coordinate frame relative to patient anatomy.

In some embodiments, the processor is further configured to fuse video data from the vision module with navigational data, generate one or more guidance views, and present the instrument relative to preoperative and/or intraoperative patient data, including by way of example CT data, MRI data, multiplanar views, three-dimensional renderings, trajectory views, bullseye alignment views, target zones, avoidance zones, and distance-to-zone information. In some embodiments, the processor generates overlays on the live instrument video and may further perform image enhancement, dimensional annotation, anatomy recognition, segmentation, or other analytics.

In some embodiments, the system is configured to perform an initial calibration establishing one or more transforms between a fiducial coordinate frame associated with the fiducial assembly and at least one of the camera coordinate frame and the distal-working-point coordinate frame. In certain embodiments, the system is further configured to verify calibration prior to use and to perform recalibration in response to one or more conditions, including re-seating or replacement of the fiducial assembly, a reduction in visible fiducials, a tracking residual exceeding a threshold, inconsistency between tracked pose and image-derived information, or user request. In embodiments having a movable, adjustable, or selectively flexible portion between the fiducial assembly and the distal working region, the system may additionally select, confirm, or update a transform corresponding to a current instrument state.

In some embodiments, the processor is configured to determine a confidence state for tracked guidance based on one or more factors including visible fiducial subset, fit quality, image-based consistency, or persistence of occlusion. The system may responsively provide one or more warnings, degrade or suppress overlays, temporarily maintain a last valid pose estimate, reacquire tracking when fiducial visibility is restored, and optionally prompt or initiate re-verification or recalibration.

In another aspect, the invention provides a method comprising providing the above-described surgical instrument and navigation system, establishing or retrieving one or more transforms between a fiducial coordinate frame associated with the fiducial assembly and at least one of a camera coordinate frame and a distal-working-point coordinate frame, tracking the fiducial assembly during use, deriving a navigated pose of at least one of the camera coordinate frame and the distal-working-point coordinate frame, determining a confidence state, and updating displayed guidance and/or recalibration state in response to one or more detected conditions.

These and other features, aspects, and advantages of the invention will become better understood from the following drawings, description, and claims.

### BRIEF DESCRIPTION OF THE DRAWINGS

Various embodiments are herein described, by way of example only, with reference to the accompanying drawings, wherein:

FIG. 1A shows schematically a first embodiment of a surgical instrument in the form of a 90° dissector.

FIG. 1B shows schematically a second embodiment of the surgical instrument in the form of a 90° ball probe.

FIG. 1C shows schematically a third embodiment of the surgical instrument in the form of a nerve hook.

FIG. 1D shows schematically a fourth embodiment of the surgical instrument in the form of curved scissors.

FIG. 1E shows schematically a fifth embodiment of the surgical instrument in the form of a pituitary rongeur with tooth.

FIG. 1F shows schematically a sixth embodiment of the surgical instrument in the form of a straight and upbiting pituitary rongeur.

FIG. 1G shows schematically a seventh embodiment of the surgical instrument in the form of a downbiting pituitary rongeur.

FIG. 1H shows schematically an eighth embodiment of the surgical instrument in the form of a suction tube (e.g. Frazier styled suction).

FIG. 2A shows a close-up view of an ninth embodiment of the surgical instrument.

FIG. 2B shows a close-up view of a tenth embodiment of the surgical instrument.

FIG. 2C shows a close-up view of a eleventh embodiment of the surgical instrument.

FIG. 2D shows a close-up view of a twelfth embodiment of the surgical instrument.

FIG. 3A shows an example of the use of the surgical instrument.

FIG. 3B shows a first example view of an image obtained from the vision module.

FIG. 3C shows a second example view of an image obtained from the vision module.

FIG. 3D shows a third example view of an image obtained from the vision module.

FIG. 4A shows schematically a first embodiment of coupling the vision module with the body of the surgical instrument.

FIG. 4B shows schematically a second embodiment of coupling the vision module with the body of the surgical instrument.

FIG. 4C shows schematically a third embodiment of coupling the vision module with the body of the surgical instrument.

FIG. 4D shows schematically a fourth embodiment of coupling the vision module with the body of the surgical instrument.

FIG. 5 shows a functional diagram of a surgical system.

FIG. 6 shows a perspective view of an embodiment of a proximal fiducial assembly mounted on a proximal handle and/or shaft region of the surgical instrument.

FIG. 7 shows a flowchart of a method for calibration, verification, tracking, confidence assessment, and recalibration of the surgical instrument during use.

### DETAILED DESCRIPTION

The following detailed description is of the best currently contemplated modes of carrying out the invention. The description is not to be taken in a limiting sense, but is made merely for the purpose of illustrating the general principles of the invention.

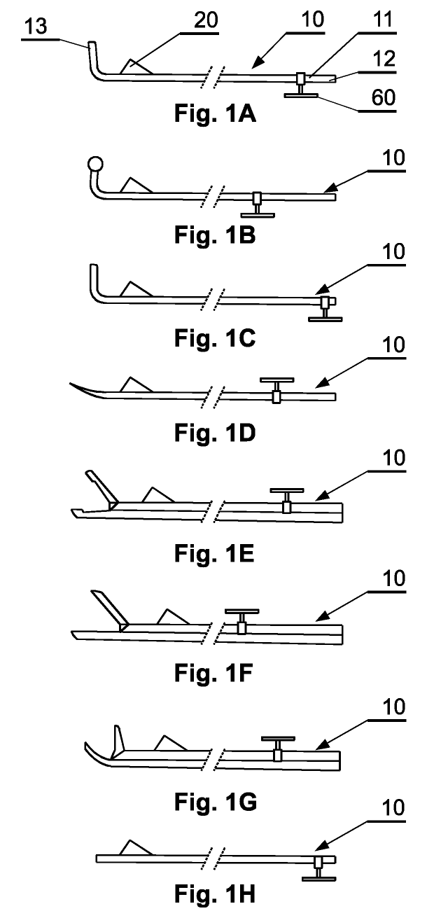

Referring now to FIGS. 1A-7, exemplary embodiments of a surgical instrument 10 and associated surgical systems are described. Unless the context requires otherwise, the term “proximal” refers to a portion of the instrument nearer the user, and the term “distal” refers to a portion of the instrument nearer the operative site. Likewise, a “distal working point” refers to a predetermined reference location at the distal working region used for calibration, navigation, and guidance, and a “distal working region” refers to the portion of the instrument intended to contact tissue, manipulate tissue, deliver suction, or otherwise perform a surgical task.

The predetermined distal working point may be defined differently for different instrument forms. By way of example, for a probe, hook, dissector, or similar single-tip instrument, the distal working point may be the terminal contacting point of the instrument. For a hollow instrument such as a suction tube, the distal working point may be the center of a distal opening, a point on a distal edge of the opening, or another predefined reference location associated with the distal end. For a jawed or multi-edge instrument, such as scissors or a rongeur, the distal working point may be a tip of a selected jaw, a midpoint between jaw tips in a defined reference state, or another predefined reference location associated with the distal working geometry.

Where useful for guidance, the system may also define a distal working axis W associated with the distal working region. The distal working axis W may coincide with a centerline of a suction opening, an axis of advance of a distal tip, an axis normal to a defined jaw reference state, or another predetermined direction associated with the distal working geometry. The longitudinal axis of the elongated body is designated X, and the optical axis of the camera is designated O. Unless otherwise stated, trajectory guidance may be referenced to the distal working axis W, the camera optical axis O, or both, while structural description of the instrument body may be referenced to the longitudinal axis X.

The surgical instruments 10 generally comprises a body 11 extending from a proximal region 12 to a distal region 13. The body 11 may be rigid, semi-rigid, or selectively flexible depending on the intended procedure and instrument type. In embodiments in which bending, articulation, or other motion occurs between the fiducial assembly and the distal working region during use, the instrument may further include one or more constraints, sensors, encoders, commanded-state definitions, or other state information such that the spatial relationship between the proximal fiducial assembly and the distal working region is fixed for a defined operating state or is otherwise known or derivable for a current operating state. In some embodiments, the body 11 is formed from one or more medical-grade materials such as stainless steel, titanium, high-strength polymers, carbon-fiber composites, biocompatible ceramics, or combinations thereof. The body 11 may be formed as a single integral member or as an assembly of multiple joined components.

The proximal region 12 is configured to be held by a surgeon and, in some embodiments, may be configured for coupling to a holder, articulated arm, robotic arm, or other support structure. The proximal region 12 may include ergonomic features such as a textured grip, finger rests, anti-slip surfaces, enlarged grip portions, contouring matched to hand position, or integrated control surfaces. In some embodiments, the proximal region 12 may also house one or more components associated with operation of the instrument 10, including by way of example a controller, a battery, a communication interface, one or more cables, and/or one or more navigation-related components described further below.

The distal region 13 defines the operative portion of the instrument 10 and may be configured in a wide range of forms depending on the intended surgical task. The distal region 13 may have a tapered, blunt, rounded, serrated, curved, angled, hooked, jawed, or hollow configuration. For each instrument type, the distal working point and, where applicable, the distal working axis W may be predefined relative to the distal geometry for calibration and navigation purposes.

A vision module 20 is combined with the body 11 and is configured to acquire visual data from tissue adjacent the distal region 13. In some embodiments, the vision module 20 is integrated into the body 11. In other embodiments, the vision module 20 is attached to the body 11 as a separate module. The vision module 20 may be carried at or near the distal region 13, while remaining proximal to the distal-most working point so that the optics are protected from direct contact and debris while still viewing the operative interface between the instrument and tissue.

The vision module 20 may include a housing 21 that at least partially encloses one or more subcomponents of the vision module 20. The housing 21 may be formed from one or more biocompatible materials, including medical-grade plastics, stainless steel, titanium, or combinations thereof. In some embodiments, the housing 21 is permanently integrated into the body 11 during manufacture. In other embodiments, the housing 21 is attached to the body 11 as a separate structure, for example by threaded engagement, fasteners, snap engagement, adhesive bonding, welding, brazing, or other attachment arrangements.

The vision module 20 further comprises at least one camera 22 configured to capture still images and/or live video of tissue adjacent the distal region 13. The camera 22 may be a high-resolution digital camera, such as a CCD or CMOS sensor, and in some embodiments may be a high-resolution color camera. The camera 22 may have one or more dimensions on the order of about 1 mm, for example about 1×1 mm, and in some embodiments may be smaller than about 1 mm in one or more dimensions. The camera 22 may be configured with fixed-focus optics or, in some embodiments, with variable optical settings, provided that camera calibration parameters corresponding to the active setting are known or determinable. In some embodiments, the camera 22 is mounted at a fixed angular relationship relative to the body 11. In other embodiments, the camera 22 may be mounted at an adjustable angular relationship relative to the body 11, provided that the current angular state is constrained, sensed, or otherwise determined so that the camera coordinate frame remains known or derivable during use. The camera 22 may further include infrared imaging capability, fluorescence imaging capability, or combinations thereof.

In some embodiments, the vision module 20 includes a single camera 22. In other embodiments, the vision module 20 includes multiple cameras 22, for example first and second cameras arranged to provide stereoscopic imaging, depth estimation, or local three-dimensional reconstruction of tissue adjacent the distal region 13. The relative spacing, angular relationship, and optical calibration of multiple cameras may be fixed at manufacture or determined during a calibration process. The first and second cameras may be carried in a common distal housing or in adjacent housings fixed relative to one another, and may have a predetermined baseline and angular relationship selected for local depth estimation in the region of the distal working point.

The camera 22 is positioned proximal to the distal-most working point of the instrument 10 by a predetermined offset distance D. In some embodiments, the distance D is selected to reduce direct contact between the camera 22 and tissue, blood, irrigation fluid, or debris while still maintaining the distal working point and adjacent anatomy within the field of view 3 of the camera 22. By way of example, the distance D may be between about 3 mm and about 15 mm. In some embodiments, the distance D may be between about 5 mm and about 15 mm. Representative embodiments include a distance D of about 6 mm and a distance D of about 12 mm, although other values may be used.

The native captured field of view 3 of the camera 22 may be selected to encompass the distal working point, a fragment of the distal region 13, and tissue immediately adjacent the distal region 13. In some embodiments, the distal region 13 occupies no more than about 50% of the native captured field of view 3. In further embodiments, the distal region 13 occupies no more than about 40%, no more than about 30%, no more than about 20%, no more than about 15%, no more than about 10%, or no more than about 5% of the native captured field of view 3. In certain embodiments, the distal region 13 occupies less than about 20% of the image area, and in one representative use case occupies about 8% of the native captured field of view 3, thereby maximizing visibility of surrounding anatomy. In some embodiments, the optical axis O of the camera 22 forms an angle α with respect to the longitudinal axis X of the instrument 10. The angle α may be fixed. In embodiments in which the angle α is adjustable, a corresponding camera model and spatial relationship for the active angular state may be stored, selected, or determined during use.

One or more light sources 23 are provided to illuminate the field of view 3. In some embodiments, the light source 23 comprises one or more light-emitting diodes (LEDs) positioned adjacent the camera 22, for example around the camera 22 or beside the camera 22. The LEDs may provide adjustable brightness, and may emit visible light, infrared light, ultraviolet light, or selected wavelength bands configured to enhance visibility of particular tissues or structures. In some embodiments, an LED may have a footprint as small as about 0.2×0.2 mm.

In embodiments where the light source 23 is located near the camera 22, a separator 23S may be provided between the camera 22 and the light source 23 to reduce light bleeding, flare, or optical interference. The separator 23S may be an opaque wall, baffle, coating, shield, or other light-management structure.

A controller 24 may be provided to manage operation of the vision module 20, including the camera 22 and the light source 23. The controller 24 may be located within the housing 21, at the proximal region 12, or at another location within the instrument 10 or an associated system. The controller 24 may perform one or more functions such as image pre-processing, compression, exposure control, white-balance control, synchronization of image capture and illumination, or communication management.

A battery 25 may be provided to power one or more components of the instrument 10 and/or the vision module 20. The battery 25 may be located within the housing 21, within the proximal region 12, or external to the instrument 10. In some embodiments, the battery 25 is rechargeable and may support inductive or wireless charging. In other embodiments, power may be supplied through a wired connection from an external power source.

A communication module 27 may be provided for transmitting data to and/or from the instrument 10. The communication module 27 may comprise a wired interface, such as a USB or other cable-based interface, and/or a wireless interface using Wi-Fi, Bluetooth, radiofrequency communication, infrared communication, or proprietary communication protocols. In some embodiments, the communication module 27 transmits image data from the camera 22 to a data processing module 30 and receives control signals for operation of the vision module 20.

Where one or more components of the vision module 20 are physically separated from one another, one or more cables 28 may couple the separated components. The cable 28 may be routed within the body 11, along the outer periphery of the body 11, or within a dedicated duct 29 formed on or within the body 11. The duct 29 may protect the cable 28 from damage and reduce interference with surgical manipulation of the instrument 10. In some embodiments, the duct 29 is accessible for maintenance, replacement, or assembly.

In some embodiments, the housing 21 contains substantially all functional components of the vision module 20, including the camera 22, one or more light sources 23, the controller 24, the battery 25, and the communication module 27. In other embodiments, the housing 21 contains only a subset of such components, for example the camera 22 and one or more light sources 23, while the controller 24, battery 25, communication module 27, and/or other components are located in the proximal region 12. Such distributed arrangements may reduce the size of the distal housing 21 and improve the balance or ergonomics of the instrument 10.

The instrument 10 may be used as a conventional hand-held surgical instrument or may be mounted to or cooperatively controlled by a robotic system. In either arrangement, the instrument 10 may provide live visual feedback from tissue adjacent the distal region 13 and may further cooperate with one or more external systems, including by way of example one or more image-processing systems, navigation systems, display systems, and data sources.

The instrument 10 may be used in a surgical system that further includes a data processing module 30 configured to receive and process data from the vision module 20. The data processing module 30 may be implemented as a dedicated computer located in an operating room, as an embedded processor within another surgical platform, as part of a robotic surgery system, or as a remote or distributed computing environment. The data processing module 30 may communicate with the instrument 10 through wired and/or wireless interfaces.

The data processing module 30 may include a communication interface 31, a data processor 32, and, in some embodiments, one or more specialized processing modules such as an artificial intelligence module 33. The communication interface 31 may receive image data from the camera 22 and may transmit control or power signals to the instrument 10. The data processor 32 may be implemented using one or more CPUs, GPUs, FPGAs, ASICs, or other programmable or dedicated computing devices.

The data processor 32 may perform one or more image-processing operations on image data received from the camera 22. Such operations may include cropping, resizing, distortion correction, sharpening, contrast enhancement, noise reduction, color correction, and stabilization of a displayed image derived from the camera output. In some embodiments, the user may select a displayed subregion 3′ of the native captured field of view 3 using an overlay box 53 or another selection interface, whereupon the selected subregion is displayed as a cropped or otherwise processed image. The displayed subregion 3′ is a displayed portion of the native captured field of view 3 and need not correspond to a change in the optical field of view of the camera 22.

The data processor 32 may also generate dimensional or contextual information for presentation with the camera image. For example, a dimensional marker 54 and optional textual representation 55 may be generated based on recognition of a known feature of the instrument 10 or another known reference in the image. Such overlays may be selectively activated or deactivated according to user preference or workflow.

In some embodiments, the artificial intelligence module 33 performs one or more advanced analytics functions, including anatomy recognition, segmentation, annotation, tissue classification, landmark identification, and image enhancement. Such functions may use machine-learning or deep-learning models trained on medical images and may operate on image data from the camera 22 alone or in combination with additional information from external data sources 43.

External data sources 43 may provide preoperative and/or intraoperative data that can be used to augment operation of the system. By way of example, the external data sources 43 may include CT data, MRI data, fluoroscopic data, ultrasound data, patient records, planned trajectories, anatomical models, and procedure-specific metadata. Such data may be used to provide the operator with contextual information, guidance, or augmented visualizations during a procedure.

Visual feedback may be presented on an external display 41, such as a medical-grade monitor, touch display, microscope display, head-up display, or wearable display. User interaction may be provided by a user interface 42, which may include a keyboard, pointer, touch interface, voice interface, haptic interface, foot pedal, instrument-mounted controls, or combinations thereof. Through the user interface 42, the operator may control camera settings, field-of-view settings, illumination settings, display layouts, overlays, and other system functions.

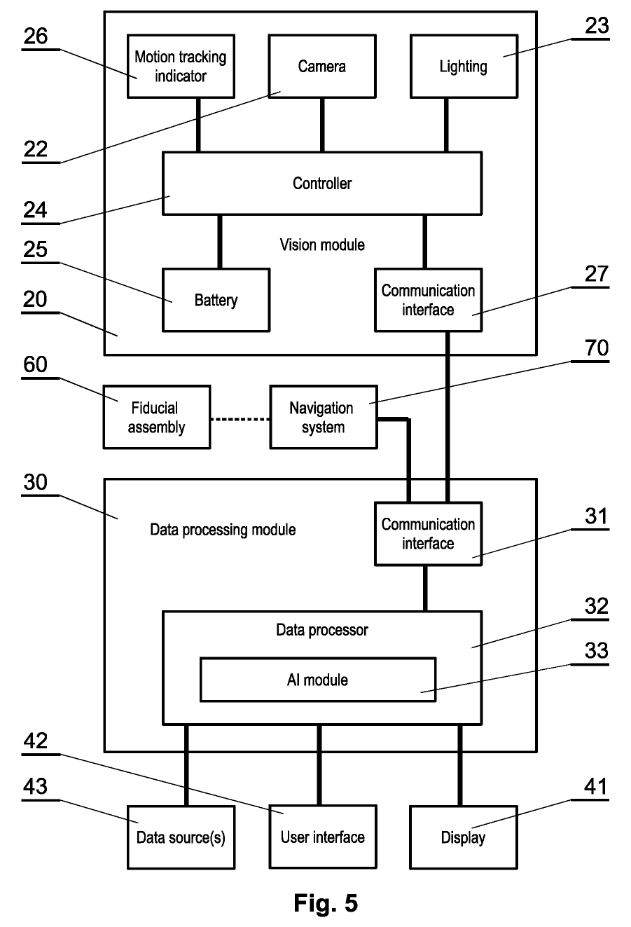

In some embodiments, the data processing module 30 is configured to combine live visual data from the vision module 20 with additional positional, anatomical, or guidance information so as to improve situational awareness during a procedure. Detailed examples of fiducial-based tracking, transform management, calibration, recalibration, confidence assessment, and guidance presentation are described further below in connection with FIGS. 5-7.

FIGS. 1A-1H illustrate representative forms of the surgical instrument 10. These figures are exemplary only and are not limiting. The body 11 may have a form such as a 90° dissector (FIG. 1A), a 90° ball probe (FIG. 1B), a nerve hook (FIG. 1C), curved scissors (FIG. 1D), a pituitary rongeur with tooth (FIG. 1E), a straight and upbiting pituitary rongeur (FIG. 1F), a downbiting pituitary rongeur (FIG. 1G) and a suction tube (FIG. 1H). Other instruments, such as a Woodson dissector or Penfield dissector, can be provided in an equivalent manner.

In the embodiments of FIGS. 1A-1H, the vision module 20 is disposed at or near the distal region 13 such that the camera 22 captures a real-time view of the operative site as the instrument 10 is manipulated. The captured image may be displayed on the external display 41 to provide an enlarged or otherwise enhanced view of anatomy that may be difficult to see directly through a small access corridor.

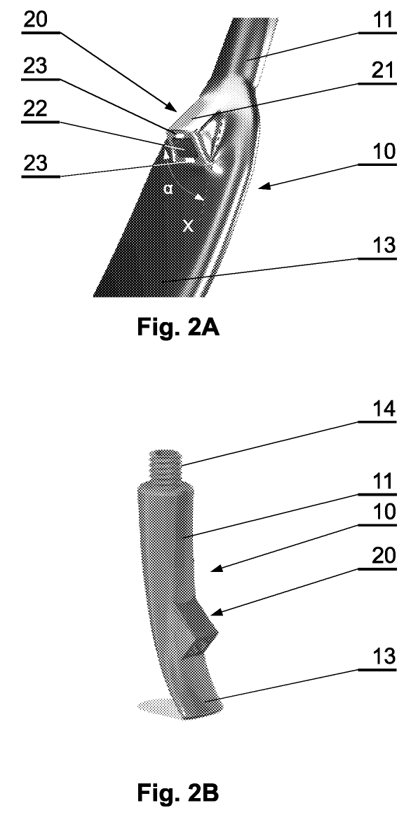

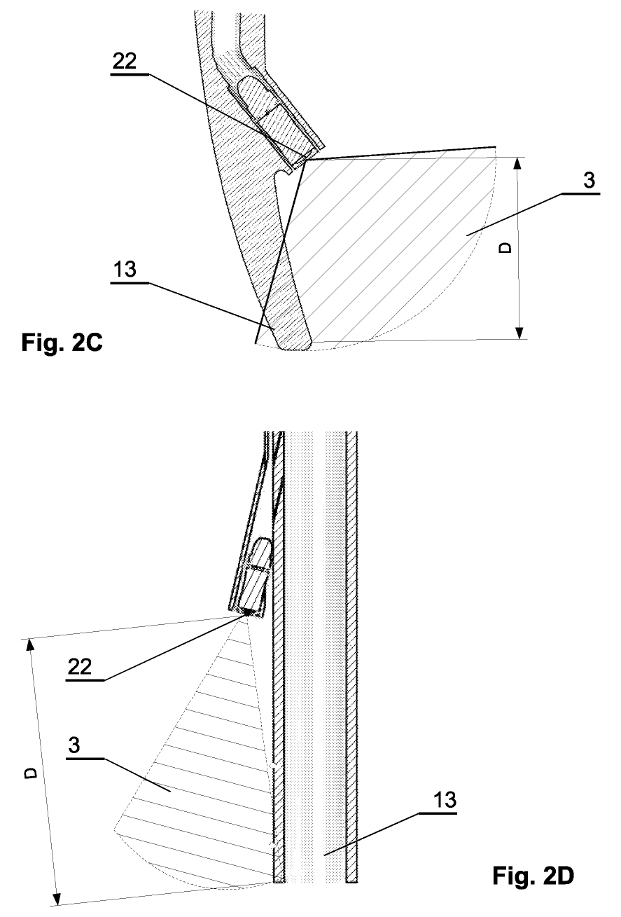

FIG. 2A shows an embodiment in which the housing 21 of the vision module 20 is integrated with the body 11 of the instrument 10. The housing 21 contains the camera 22 and a plurality of light sources 23 positioned adjacent the camera 22 so as to illuminate the field of view 3. In some embodiments, the angle α of the line of sight of the camera 22 relative to the longitudinal axis X of the instrument 10 may be selected according to the procedure.

FIG. 2B shows an embodiment in which the body 11 includes a distal tool portion configured as a screw-on or otherwise attachable tool that may be coupled by a threaded end 14 or other connector to a proximal carrier, handle, robotic arm interface, or other support element. The vision module 20 may be integrated into the attachable tool or secured thereto. In such embodiments, the surgical instrument 10 may comprise the distal tool portion together with the proximal carrier or handle when assembled, and the fiducial assembly 60 may be mounted to the proximal carrier or handle, to another proximal portion of the assembled instrument, or to another structure having a fixed or known spatial relationship to the distal tool portion.

FIG. 2C shows one representative embodiment in which the camera 22 is positioned at a distance D of about 6 mm from the distal working point and has an angular field of view of about 90°. FIG. 2D shows another representative embodiment, here in the form of a suction tube, in which the camera 22 is positioned at a distance D of about 12 mm from the distal working point and has an angular field of view of about 40°. These values are illustrative and may be varied in other embodiments.

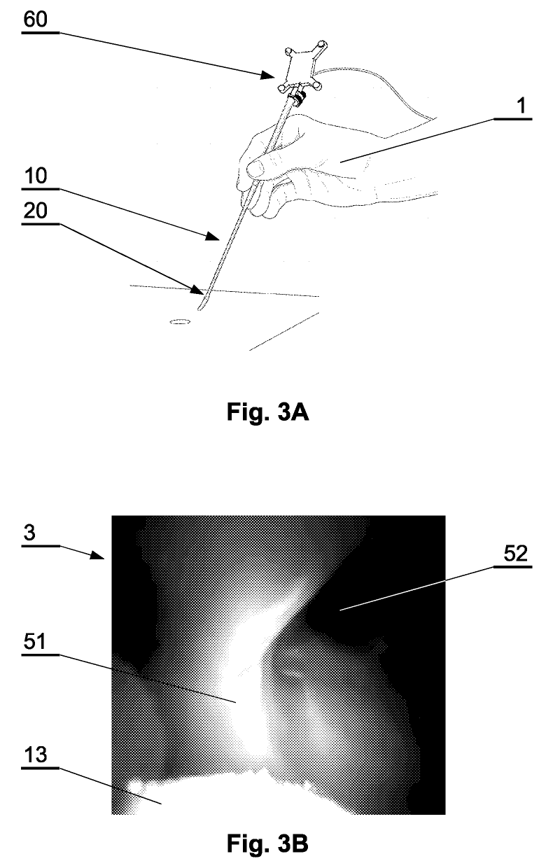

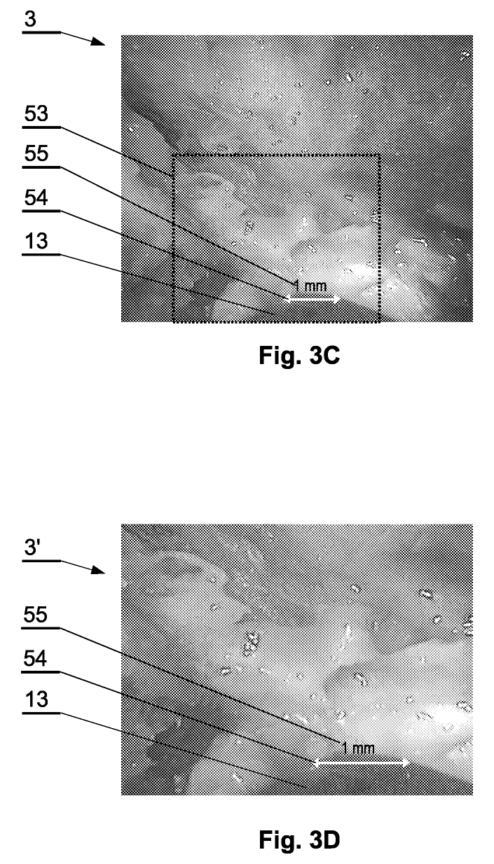

FIGS. 3A-3D illustrate representative use of the instrument 10 and representative video presentations. As the operator manipulates the instrument 10, the vision module 20 illuminates tissue near the distal region 13 and captures image data from the local field. In one representative view, the distal region 13 occupies only a small portion of the field of view 3, thereby preserving visibility of adjacent anatomy.

In some embodiments, a first displayed image may correspond to the native captured field of view 3, while a later displayed image corresponds to a user-selected displayed subregion 3′ of the native captured field of view 3. Selection of the displayed subregion may be performed by use of an overlay box 53 or another selection tool. A dimensional marker 54 and/or textual representation 55 may optionally be displayed to provide scale information.

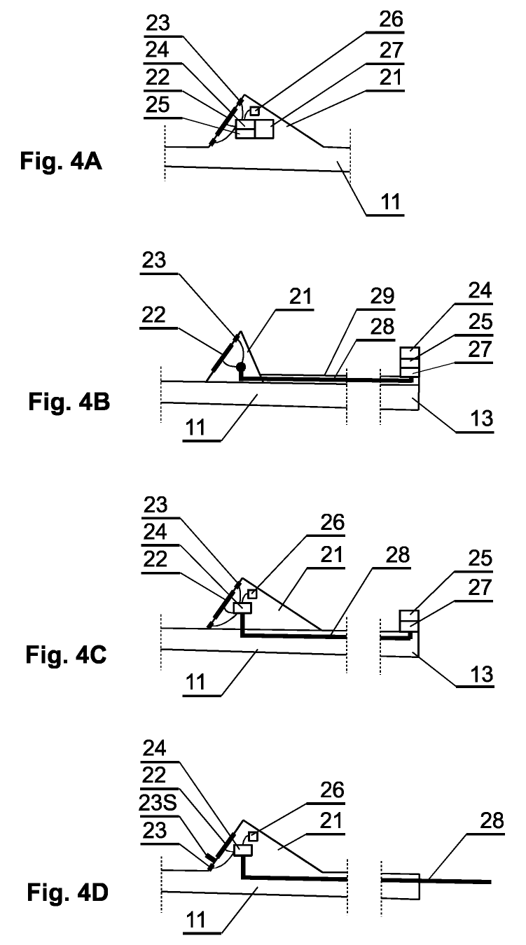

FIGS. 4A-4D illustrate representative arrangements for distributing components of the vision module 20 and associated electronics. In the embodiment of FIG. 4A, the housing 21 is integrated with the body 11 and may contain the camera 22, one or more light sources 23, the controller 24, the battery 25, and the communication module 27. This arrangement can provide a highly integrated instrument that is handled similarly to a conventional instrument of the same form factor.

In the embodiment of FIG. 4B, the housing 21 is attached to the body 11 as an attachment module and may contain, for example, the camera 22 and one or more light sources 23, while the controller 24, battery 25, and communication module 27 are located remotely, for example in the proximal region 12. In such embodiments, the cable 28 may run along an outer surface of the body 11 in a dedicated duct 29.

In the embodiment of FIG. 4C, the housing 21 is attached to the body 11 and may contain the camera 22, one or more light sources 23, and optionally one or more additional local electronics or sensing components, while one or more other components such as the battery 25 and communication module 27 are located at the proximal region 12. The cable 28 may extend within the body 11 to reduce external clutter.

In the embodiment of FIG. 4D, the housing 21 is integrated with the body 11 and may contain the camera 22, multiple light sources 23, the controller 24, and optionally additional electronics. A separator 23S may be positioned between the camera 22 and one or more light sources 23. Power and/or data may be communicated through the cable 28 to the data processing module 30 or another external system.

It will be appreciated that the embodiments shown in FIGS. 1A-4D are exemplary and that features from different embodiments may be combined. Likewise, features described in connection with one instrument form may be applied to other instrument forms, and features described in connection with one component-distribution arrangement may be applied to another arrangement.

The surgical instrument 10 further comprises a fiducial assembly 60 mounted at or near the proximal region 12 of the body 11 and configured to be detected by an external navigation system 70. The fiducial assembly 60 provides a tracked reference structure located proximally of the distal region 13 while preserving direct local visualization at the distal region 13 by the vision module 20.

In some embodiments, the fiducial assembly 60 comprises a support structure 61 carrying a plurality of optical tracking elements 62 arranged in a predefined geometry. The support structure 61 may be configured as a low-profile, lightweight frame, bridge, collar, cap, ring, armature, dorsal arch, lateral cantilever, proximal extension, or other mounting structure selected to reduce obstruction of instrument handling while maintaining visibility to the external navigation system 70.

The optical tracking elements 62 may comprise passive retroreflective markers, active infrared emitters, illuminated fiducials, patterned optical fiducials, or combinations thereof. In some embodiments, the fiducial assembly 60 is optimized for optical infrared tracking. In further embodiments, the fiducial assembly 60 may additionally include one or more components supporting other tracking modalities, such as electromagnetic or radiofrequency tracking, while still functioning as an optically trackable fiducial assembly.

The optical tracking elements 62 may be arranged to provide reliable six-degree-of-freedom pose determination. In some embodiments, at least three non-collinear optical tracking elements 62 are provided. In further embodiments, four, five, six, or more optical tracking elements 62 are provided. The geometry may be planar, partially elevated, or non-coplanar. Representative arrangements include, by way of example, an asymmetric triangular arrangement, a skewed quadrilateral arrangement, a Y-shaped frame with one elevated optical tracking element, and a cluster having a non-coplanar or tetrahedral relationship among at least a subset of the optical tracking elements 62.

In some embodiments, the geometry of the optical tracking elements 62 is selected to reduce ambiguity in pose estimation, improve visibility from multiple viewing angles, and preserve trackability even when one or more optical tracking elements 62 are temporarily obscured. The geometry may be asymmetric to discourage rotational ambiguity and may be selected such that at least a validated subset of the optical tracking elements 62 remains usable for pose estimation under partial occlusion.

The fiducial assembly 60 may be mounted at a variety of locations at or near the proximal region 12. By way of example, the fiducial assembly 60 may be mounted on a proximal handle cap, a proximal shaft collar, a dorsal bridge above a hand-engagement region, a lateral frame extending from the body 11, or a proximal extension generally aligned with the longitudinal axis X of the instrument 10. In some embodiments, the fiducial assembly 60 is positioned to remain outside the surgeon’s primary hand-contact region while maintaining line of sight to the external navigation system 70.

The fiducial assembly 60 may be permanently integrated with the body 11 or mounted to the body 11 as a removable module. In permanently integrated embodiments, the support structure 61 may be fixed to the body 11 by welding, brazing, adhesive bonding, overmolding, integral machining, fasteners, or other permanent attachment techniques. In removable embodiments, the fiducial assembly 60 may be coupled to the body 11 by a releasable mechanical interface, including by way of example threaded engagement, a bayonet interface, a latch, a clip, a snap fit, a keyed sleeve, or another removable coupling structure.

In some embodiments, sterilization considerations may influence the selected mounting arrangement. By way of example, the fiducial assembly 60 may be permanently integrated into a fully sterilizable instrument, may be removable for separate sterilization, may be provided with a sterile barrier or drape, may comprise a reusable carrier and a disposable sterile marker structure, or may be mounted by way of a sterile adapter coupled to the proximal region 12.

One or more reference features 63 may be provided by cooperation between the fiducial assembly 60 and the body 11, or, in permanently integrated embodiments, by manufactured reference geometry fixed between the fiducial assembly 60 and the body 11, so as to establish a repeatable spatial relationship between the fiducial assembly 60 and the body 11. Such reference features 63 may include one or more datum surfaces, alignment shoulders, anti-rotation features, pins, sockets, flats, slots, grooves, tongues, keyed bayonet features, splines, polygonal hubs, asymmetric fastener patterns, machined reference surfaces, or combinations thereof.

In some embodiments, the reference features 63 establish a repeatable seating orientation of the fiducial assembly 60 relative to the body 11. In removable embodiments, the reference features 63 may constrain translational and rotational degrees of freedom upon attachment such that reseating of the fiducial assembly 60 produces a repeatable pose relative to the body 11 within a predetermined verification or calibration tolerance. In permanently integrated embodiments, the reference features 63 may be embodied by fixed manufacturing relationships, datum geometry, or other structural relationships established during manufacture and thereafter used for calibration, even though no user-actuated reseating occurs during normal use.

In some embodiments, the fiducial assembly 60 and the body 11 further comprise one or more auxiliary interface features, such as electrical contacts, optical couplings, magnetic couplings, position switches, identification features, or memory elements. Such auxiliary interface features may provide power to active optical tracking elements 62, may identify a particular fiducial assembly 60 to the data processing module 30, may indicate whether the fiducial assembly 60 is properly seated, and/or may enable retrieval of calibration data associated with a particular fiducial assembly 60 or instrument 10.

The external navigation system 70 may comprise one or more external tracking cameras, sensors, emitters, processors, and/or navigation workstations configured to detect the optical tracking elements 62 and to generate marker observations and/or determine a pose of the fiducial assembly 60 in a navigation coordinate frame. In some embodiments, the external navigation system 70 detects reflected infrared light from passive markers. In other embodiments, the external navigation system 70 detects emitted light from active markers. The external navigation system 70 may provide a determined fiducial pose to the data processing module 30, or the data processing module 30 may receive marker observations and derive or refine the fiducial pose. The external navigation system 70 may also participate in patient registration, or may cooperate with a separate workstation or the data processing module 30 so as to establish a relationship between the navigation coordinate frame and patient anatomy.

In some embodiments, the system defines a fiducial coordinate frame FF fixed relative to the fiducial assembly 60, a camera coordinate frame CF fixed relative to the camera 22, and a distal-working-point coordinate frame TF fixed relative to the distal working region. In some embodiments, the origin of the camera coordinate frame CF is located at or referenced to the camera optical center and one axis of the camera coordinate frame CF is aligned with the camera optical axis O. In some embodiments, the origin of the distal-working-point coordinate frame TF is located at the predetermined distal working point, and one axis of the distal-working-point coordinate frame TF is aligned with the distal working axis W when such an axis is defined. In certain embodiments, the system further defines a patient or anatomy frame PF and, where applicable, a stereo reconstruction frame SF associated with a multi-camera embodiment of the vision module 20.

One or more transforms are established and stored so as to define the spatial relationship between the fiducial assembly 60 and the distal visual and/or working geometry of the instrument 10. By way of example, a first transform may relate the fiducial coordinate frame FF to the camera coordinate frame CF, and a second transform may relate the fiducial coordinate frame FF to the distal-working-point coordinate frame TF. In embodiments having fixed geometry between the fiducial assembly 60 and the distal working region, such transforms may be fixed for the instrument. In embodiments having articulated, adjustable, or selectively flexible geometry between the fiducial assembly 60 and the distal working region, a respective transform may be associated with each defined instrument state, or a transform may be updated according to sensed or otherwise determined state information. In use, a detected pose of the fiducial coordinate frame FF, together with a registration between the navigation coordinate frame and the patient frame PF and together with the relevant transform or transforms, is sufficient to compute a current pose of the camera coordinate frame CF and/or the distal-working-point coordinate frame TF in the patient frame PF.

In some embodiments, the transform between the fiducial assembly 60 and the camera 22 and/or distal working point is determined by an initial calibration process. The initial calibration process may be performed during manufacture, during instrument setup prior to surgery, after attachment of a removable fiducial assembly 60, or at another suitable time.

In one representative embodiment, a factory calibration is performed after final assembly of the instrument 10 to determine the relative position and orientation of the fiducial assembly 60, the camera 22, and the distal working point. Factory calibration data may be stored in a memory associated with the instrument 10, the fiducial assembly 60, the data processing module 30, or another component of the system.

In some embodiments, the system performs a pre-use verification procedure before the start of surgery. During pre-use verification, the fiducial assembly 60 may be detected by the external navigation system 70 while the camera 22, the distal working point, or both, are positioned relative to a known reference object, calibration target, calibration block, or fixture. The measured relationship may be compared against stored calibration data to determine whether a previously stored transform remains valid. If deviation exceeds a selected criterion, the system may prompt recalibration, limit one or more overlay functions, or indicate a reduced-confidence state. A verification procedure may confirm continued suitability of a stored transform without necessarily replacing the stored transform.

In some embodiments, calibration and/or recalibration may be performed using a fixture-based procedure. For example, the distal region 13 may be placed in contact with or adjacent to one or more known reference features of a calibration jig while the fiducial assembly 60 is simultaneously tracked by the external navigation system 70. In other embodiments, a touch-to-known-point calibration may be performed in which the distal working point is brought into contact with one or more known spatial points.

In some embodiments, image-based calibration and image-based verification may additionally or alternatively be used. For example, the camera 22 may image a known calibration pattern, a known fiducial target, or one or more known geometric features. In an image-based calibration procedure, the observed image data may be used to determine or update camera intrinsic parameters, camera pose, and/or a relationship between the camera coordinate frame CF and the fiducial coordinate frame FF. In an image-based verification procedure, the observed image data may be used to assess whether stored calibration parameters or stored transforms remain within an acceptance criterion without necessarily replacing the stored calibration parameters or stored transforms.

In embodiments having multiple cameras 22, calibration may further include determination of one or more camera-to-camera transforms, baseline spacing, relative angular orientation, and intrinsic/extrinsic camera parameters. Such calibration may be used to support stereoscopic imaging, local depth estimation, or local three-dimensional reconstruction of tissue near the distal region 13.

During surgical use, the external navigation system 70 determines, or provides data from which is determined, a pose of the fiducial assembly 60 in a navigation coordinate frame, and the data processing module 30 applies a registration between the navigation coordinate frame and the patient frame PF together with one or more stored transforms to derive the pose of the camera coordinate frame CF and/or distal-working-point coordinate frame TF in the patient frame PF. This derived pose may be used to localize the live video feed to one or more anatomical landmarks, planned trajectories, anatomical models, and/or preoperative or intraoperative image data.

In some embodiments, the data processing module 30 generates one or more navigational views based on the derived pose. Such navigational views may include axial, sagittal, coronal, oblique, trajectory-aligned, or three-dimensional rendered views of patient anatomy, together with a virtual representation of the instrument 10 and/or the distal working point. In some embodiments, an “instrument-eye” or “bullseye” view is generated to indicate alignment of the distal working axis W, the camera optical axis O, or both, with a planned trajectory.

In some embodiments, the system enables surgical planning and guidance relative to one or more target zones and/or avoidance zones defined within a patient model. The data processing module 30 may calculate distances from the origin of the distal-working-point coordinate frame TF, from the distal working axis W, from the camera optical axis O, or from another relevant instrument feature to a target zone or avoidance zone and may generate visual, audible, and/or haptic alerts when the instrument 10 approaches a selected boundary condition.

In some embodiments, the data processing module 30 generates augmented overlays on the live video obtained from the camera 22. By way of example, the overlays may include a planned trajectory, target markers, outlines of anatomical structures, depth indicators, distance-to-zone indicators, color-coded safety regions, confidence indicators, labels, segmentation boundaries, or combinations thereof. Such overlays may reduce the need for the operator to look away from the live local view to consult a separate navigation display.

In some embodiments, the system further employs one or more advanced analytics modules, including artificial intelligence or machine-learning modules, to recognize, classify, segment, annotate, or otherwise analyze anatomical structures present in the live video feed. Such image-derived information may be correlated with the known navigated pose of the instrument 10 to provide enhanced contextual awareness, decision support, and consistency checking.

In embodiments having multiple cameras 22, the data processing module 30 may determine local depth data or local three-dimensional surface information from stereoscopic image pairs. Such locally derived three-dimensional information may be displayed directly to a user and/or may be used to verify registration, refine overlays, verify proximity of the distal working point to a visible structure, or improve confidence assessment.

The system may be configured to determine a confidence state associated with tracked guidance. The confidence state may be based on one or more factors including, by way of example, a number of optical tracking elements 62 currently visible to the external navigation system 70, geometric conditioning of the currently visible marker subset, fit residual of a pose-estimation solution, temporal stability of pose estimates, signal quality, visibility persistence, attachment or seating status of the fiducial assembly 60, and/or inconsistency between tracked pose and image-derived information.

In some embodiments, a plurality of confidence states is defined, such as a full-confidence state, a warning state, and a reduced-confidence or suppress-overlay state. By way of non-limiting example, in embodiments using four or more optical tracking elements 62, a full-confidence state may correspond to visibility of a validated full set or designated high-confidence subset of optical tracking elements 62 and a fit residual below about 1.0 mm. A warning state may correspond to visibility of a minimum acceptable subset and/or a fit residual between about 1.0 mm and about 2.0 mm. A reduced-confidence or suppress-overlay state may correspond to visibility below the minimum acceptable subset, a fit residual above about 2.0 mm, or another selected inconsistency criterion. Other thresholds and state definitions may be used.

The confidence state may influence how guidance is presented. In a full-confidence state, the data processing module 30 may present full navigational overlays and guidance cues. In a warning state, the system may present one or more caution indicators, may reduce opacity or extent of displayed overlays, may switch to a simplified guidance display, or may prompt the user to verify continued suitability of tracked guidance. In a reduced-confidence or suppress-overlay state, the system may suppress one or more overlays, may present a warning or alarm, and/or may request recalibration or re-verification.

In some embodiments, the system is configured to tolerate transient occlusion of one or more optical tracking elements 62. For a brief interval following loss of one or more optical tracking elements 62, the system may maintain a last valid pose estimate, optionally together with a visible reduced-confidence indication. If marker visibility is restored within a selected interval and the resulting pose estimate satisfies one or more validation criteria, full or partial overlay functionality may be restored. If marker visibility is not restored or validation criteria are not satisfied, the system may maintain a degraded-guidance mode, transition to a video-only mode, or prompt recalibration. The validation criteria may include visibility of a designated minimum subset of optical tracking elements 62, a pose-estimation residual below a threshold, and agreement with a last valid pose estimate within a selected tolerance.

Recalibration and/or re-verification may be triggered automatically or manually. Triggering conditions may include, by way of example, reseating or replacement of the fiducial assembly 60, an attachment-state change, loss of marker visibility below a selected threshold, fit residual exceeding a threshold, impact or drop detection, deviation between tracked pose and image-derived landmark information, elapsed time, user request, or another event suggesting that a stored transform should be rechecked. In embodiments having articulated, adjustable, or selectively flexible geometry between the fiducial assembly 60 and the distal working region, triggering conditions may additionally include failure to confirm a current instrument state or detection of an instrument state inconsistent with a selected transform or camera model.

In some embodiments, automatic recalibration routines are available if the navigation system detects deviation from expected positions. Such response may include repeating all or part of a fixture-based calibration, a touch-to-known-point calibration, or an image-based calibration, and may optionally begin with a re-verification routine or a pre-use verification-type routine. In some embodiments, the system may first attempt a re-verification and may only proceed to a fuller recalibration if re-verification fails. Following successful recalibration, or following successful confirmation or selection of a transform corresponding to a current instrument state, the updated or confirmed transform may be stored in memory and thereafter used for continued derivation of pose of the camera coordinate frame CF and/or distal-working-point coordinate frame TF.

In removable embodiments of the fiducial assembly 60, the system may store and retrieve calibration information specific to a given fiducial assembly 60, instrument 10, or instrument-fiducial pairing. For example, a module identifier or memory element may indicate which transform set is associated with a currently attached fiducial assembly 60. Where a fiducial assembly 60 is removed and reattached, the system may require a verification or recalibration step before restoring a full-confidence guidance state.

The system may be configured to interoperate with one or more existing navigation platforms, including commercially available optical navigation platforms, while remaining platform-agnostic in architecture. In some embodiments, the fiducial assembly 60 is optimized for optical infrared tracking and is detected by an external navigation camera system without requiring material modification of the underlying navigation workflow beyond calibration and registration appropriate to the instrument 10.

Referring now to FIG. 5, the surgical system may comprise the instrument 10, the external navigation system 70, the data processing module 30, the external display 41, the user interface 42, and one or more external data sources 43. The external navigation system 70 provides marker observations and/or fiducial pose information to the data processing module 30. The vision module 20 provides live image data to the data processing module 30. The external data sources 43 may provide patient-specific image data, planning data, anatomical models, registration data, or other context data.

In some embodiments, the data processing module 30 comprises a fiducial pose engine 34 configured to receive or derive pose of the fiducial assembly 60 in the navigation coordinate frame, a transform engine 35 configured to apply a registration between the navigation coordinate frame and the patient frame PF and to apply one or more stored transforms between the fiducial assembly 60 and the camera coordinate frame CF and/or distal-working-point coordinate frame TF, a confidence engine 36 configured to determine one or more confidence states, and a guidance engine 37 configured to generate one or more navigation views and/or overlays. One or more image-processing and analytics modules, including the artificial intelligence module 33, may cooperate with the fiducial pose engine 34, transform engine 35, confidence engine 36, and guidance engine 37.

The guidance engine 37 may combine live image data, derived pose data, and patient-specific anatomical data to produce one or more guidance outputs. Such outputs may include live video with overlays, multiplanar and three-dimensional views, bullseye alignment views, target and avoidance-zone displays, distance indicators, warning indicators, and confidence indicators.

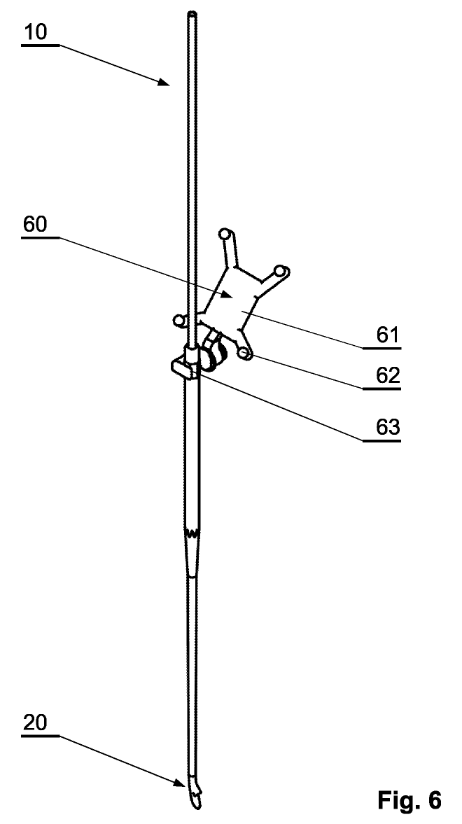

Referring now to FIG. 6, the fiducial assembly 60 is shown mounted on a proximal handle and/or shaft portion of the body 11. In the illustrated embodiment, the support structure 61 carries a plurality of optical tracking elements 62 spaced from one another in a predefined asymmetric geometry. One or more reference features 63 may be provided at an interface between the support structure 61 and the body 11 to establish a repeatable orientation and position of the fiducial assembly 60 relative to the body 11. In some embodiments, one optical tracking element 62 is elevated or otherwise offset from a plane defined by at least three other optical tracking elements 62 to improve pose observability under partial occlusion.

Although FIG. 6 illustrates one representative configuration, the fiducial assembly 60 may be mounted in many other ways consistent with the teachings herein. The support structure 61 may be more compact, more elongated, more laterally offset, or more coaxial with the body 11. Likewise, the optical tracking elements 62 may be arranged differently provided that the resulting fiducial geometry remains detectable and usable by the external navigation system 70.

Features described in connection with any embodiment may be combined with features of any other embodiment unless incompatible. For example, a removable fiducial assembly 60 may be combined with a stereoscopic vision module 20, a permanently integrated fiducial assembly 60 may be combined with fixture-based calibration and image-based verification, and any of the fiducial geometries described herein may be used with any of the instrument forms described above.

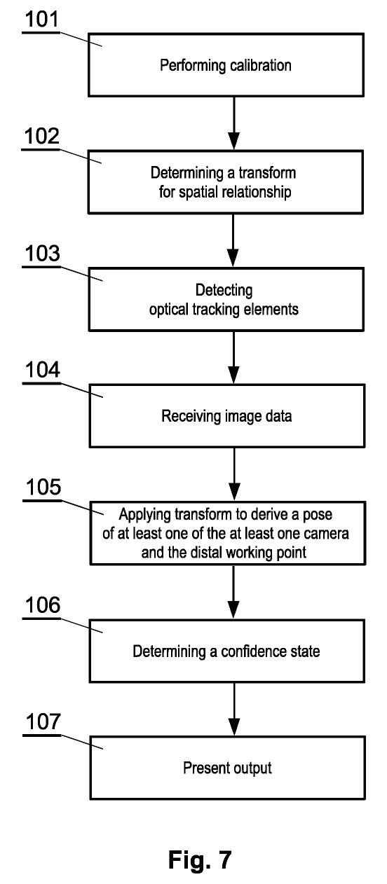

Referring now to FIG. 7, a representative method may begin with performing a calibration procedure in step 101, followed by retrieving or determining a transform corresponding to the repeatable spatial relationship in step 102. Next, the method may comprise detecting, in step 103, the optical tracking elements with an external optical navigation system and determining a pose of the fiducial assembly in a navigation coordinate frame. Image data from the at least one camera is received in step 104. The transform is applied in step 105 to derive a pose of at least one of the at least one camera and the distal working point from the pose of the fiducial assembly. In step 106, a confidence state is determined for tracked guidance based on at least one of (i) a visible subset of the optical tracking elements, (ii) a pose-estimation residual or fit quality, and (iii) consistency between the derived pose and image-derived information from the image data. Next, responsive to the confidence state, an output is presented in step 107 by: selectively (a) presenting a first guidance output, (b) presenting a warning output, or (c) suppressing at least part of the guidance output and initiating a re-verification routine or a recalibration routine.

If the confidence state satisfies a selected criterion, the method may proceed with presentation of full guidance, including by way of example one or more overlays, navigational views, and trajectory guidance cues. If the confidence state falls into a warning state, the method may present one or more caution indicators and/or simplified guidance outputs. If the confidence state falls below a selected minimum criterion, the method may suppress one or more overlays, enter a degraded-guidance mode, or initiate a recalibration or re-verification routine.

The method may further include monitoring for one or more recalibration triggers during use. Upon detection of a recalibration trigger, the method may execute a re-verification routine and, if appropriate, a recalibration routine to update one or more transforms associated with the instrument 10. The updated transform data may then be used for continued guidance presentation.

The foregoing embodiments are illustrative rather than limiting. Variations in instrument geometry, fiducial geometry, calibration workflow, confidence logic, overlay presentation, data-processing architecture, and sterilization arrangement may be made without departing from the scope of the invention as defined by the claims.

1. A surgical instrument, comprising:
an elongated body extending from a proximal region to a distal working region and defining a distal working point;
a vision module carried by the elongated body at a position proximal to the distal working point, the vision module comprising at least one camera and at least one light source;
a fiducial assembly mounted to the proximal region of the elongated body, the fiducial assembly comprising a support structure carrying a plurality of optical tracking elements arranged in a predefined geometry detectable by an external optical navigation system; and
one or more reference features defined by the fiducial assembly and the elongated body and configured to constrain relative translation and relative rotation of the fiducial assembly with respect to the elongated body so as to establish a repeatable spatial relationship between (i) a fiducial coordinate frame associated with the fiducial assembly and (ii) at least one of a camera coordinate frame of the vision module and a distal-working-point coordinate frame associated with the distal working point,
wherein the repeatable spatial relationship permits calibration of a transform between the fiducial coordinate frame and said at least one of the camera coordinate frame and the distal-working-point coordinate frame such that pose of at least one of the at least one camera and the distal working point is derivable from a detected pose of the fiducial assembly and the transform.

2. The surgical instrument of claim 1, wherein the one or more reference features comprise at least one datum surface and at least one anti-rotation feature.

3. The surgical instrument of claim 2, wherein the at least one anti-rotation feature comprises one or more of: a D-profile interface, a mating flat and slot, a pin-and-socket pair, a tongue-and-groove interface, a keyed bayonet interface, a spline, a polygonal hub, and an asymmetric fastener pattern.

4. The surgical instrument of claim 1, wherein the support structure carries at least four of the optical tracking elements in an asymmetric geometry.

5. The surgical instrument of claim 4, wherein at least one of the optical tracking elements is offset from a plane defined by at least three others of the optical tracking elements.

6. The surgical instrument of claim 1, wherein the fiducial assembly is removably attached to the elongated body and repeated mounting of the fiducial assembly to the elongated body reproduces the fiducial coordinate frame relative to the elongated body within a calibration tolerance.

7. The surgical instrument of claim 1, wherein the fiducial assembly is permanently integrated with the elongated body.

8. The surgical instrument of claim 1, wherein the vision module comprises first and second cameras fixed relative to one another and arranged to support generation of local depth data or local three-dimensional surface data adjacent the distal working region.

9. A surgical system, comprising:
a surgical instrument comprising:
an elongated body extending from a proximal region to a distal working region and defining a distal working point;
a vision module carried by the elongated body at a position proximal to the distal working point, the vision module comprising at least one camera and at least one light source;
a fiducial assembly mounted to the proximal region of the elongated body, the fiducial assembly comprising a support structure carrying a plurality of optical tracking elements arranged in a predefined geometry; and
one or more reference features defined by the fiducial assembly and the elongated body and configured to establish a repeatable spatial relationship between a fiducial coordinate frame associated with the fiducial assembly and at least one of a camera coordinate frame of the vision module and a distal-working-point coordinate frame associated with the distal working point;
an external optical navigation system configured to detect the optical tracking elements and determine a pose of the fiducial assembly in a navigation coordinate frame; and
a data processing system comprising at least one processor and memory storing instructions that, when executed by the at least one processor, cause the data processing system to:
receive image data from the at least one camera;
receive or derive the pose of the fiducial assembly from the external optical navigation system;
apply a stored transform corresponding to the repeatable spatial relationship to derive a pose of at least one of the at least one camera and the distal working point from the pose of the fiducial assembly;
determine a confidence state for tracked guidance based on at least one of (i) a visible subset of the optical tracking elements, (ii) a pose-estimation residual or fit quality, and (iii) consistency between the derived pose and image-derived information from the image data; and
generate a guidance output based at least in part on the derived pose and the confidence state.

10. The surgical system of claim 9, wherein the guidance output comprises a live video view from the at least one camera with one or more overlays superimposed thereon, the one or more overlays comprising one or more of: a planned trajectory, a target marker, an avoidance zone, a distance indicator, a confidence indicator, an anatomical structure outline, and a segmentation boundary.

11. The surgical system of claim 10, wherein, in response to a warning state, the instructions further cause the data processing system to present a caution indicator or reduce an extent, opacity, or complexity of one or more of the overlays.

12. The surgical system of claim 9, wherein, upon transient occlusion of one or more of the optical tracking elements, the instructions further cause the data processing system to maintain a last valid pose estimate for a selected interval and to restore full or partial guidance only after reacquired tracking satisfies one or more validation criteria.

13. The surgical system of claim 9, wherein the instructions further cause the data processing system to register the derived pose to a patient coordinate frame associated with preoperative or intraoperative image data, define at least one target zone or avoidance zone within a patient model, determine a distance from the distal working point or a camera optical axis to the at least one target zone or avoidance zone, and generate at least one of: a trajectory-aligned view, a bullseye alignment view, a multiplanar navigation view, a three-dimensional rendered navigation view, a visual alert, and an audible alert based on the distance.

14. A method for operating a surgical navigation system with a surgical instrument, the surgical instrument comprising an elongated body extending from a proximal region to a distal working region and defining a distal working point, a vision module carried by the elongated body at a position proximal to the distal working point, the vision module comprising at least one camera and at least one light source, and a fiducial assembly mounted to the proximal region of the elongated body, the fiducial assembly comprising a support structure carrying a plurality of optical tracking elements arranged in a predefined geometry and one or more reference features configured to establish a repeatable spatial relationship between a fiducial coordinate frame associated with the fiducial assembly and at least one of a camera coordinate frame of the vision module and a distal-working-point coordinate frame associated with the distal working point, the method comprising:
retrieving or determining a transform corresponding to the repeatable spatial relationship;
detecting the optical tracking elements with an external optical navigation system and determining a pose of the fiducial assembly in a navigation coordinate frame;
receiving image data from the at least one camera;
applying the transform to derive a pose of at least one of the at least one camera and the distal working point from the pose of the fiducial assembly;
determining a confidence state for tracked guidance based on at least one of (i) a visible subset of the optical tracking elements, (ii) a pose-estimation residual or fit quality, and (iii) consistency between the derived pose and image-derived information from the image data; and
responsive to the confidence state, selectively (a) presenting a first guidance output, (b) presenting a warning output, or (c) suppressing at least part of the guidance output and initiating a re-verification routine or a recalibration routine.

15. The method of claim 14, wherein retrieving or determining the transform comprises retrieving factory calibration data associated with the surgical instrument, the fiducial assembly, or an instrument-fiducial pairing.

16. The method of claim 14, further comprising, prior to surgery, performing a pre-use verification routine by presenting the distal working point or the at least one camera relative to a known reference object, calibration target, calibration block, or fixture and comparing a measured relationship to stored calibration data.

17. The method of claim 14, wherein retrieving or determining the transform comprises performing a fixture-based calibration procedure or a touch-to-known-point calibration procedure.

18. The method of claim 14, wherein retrieving or determining the transform comprises performing an image-based calibration or verification procedure using a calibration pattern, a fiducial target, or a known geometric feature imaged by the at least one camera.

19. The method of claim 14, further comprising detecting a re-verification or recalibration trigger based on one or more of: reseating or replacement of the fiducial assembly, an attachment-state change, a reduction in visible optical tracking elements below a threshold, a pose-estimation residual exceeding a threshold, inconsistency between tracked pose and image-derived landmark information, an impact event, elapsed time, and user request.

20. The method of claim 19, further comprising, in response to the re-verification or recalibration trigger, performing a re-verification routine before performing the recalibration routine and updating the transform when the re-verification routine fails to satisfy a selected criterion.
